(19) United States
(12) Patent Application Publication
(10) Pub. No.: US 2005/0205206 A1
(43) Pub. Date: Sep. 22, 2005

(54) Apparatus for materials processing by stimulated light emission and method of its utilization

(76) Inventor: Robert Lembersky, Bronxville Heights, NY (US)

Correspondence Address: ROBERT LEMBERSKY, 14 SIDEHILL LANE, BRONXVILLE HEIGHTS, NY, 10710-2333, US

(21) Appl. No.: 10/804,007

(22) Filed: Mar. 19, 2004

(51) Int. Cl. C23F 1/00

(52) U.S. Cl. 156/345.5; 118/723 R; 216/63

(57) ABSTRACT

A materials processing apparatus and a method of its utilization for surface modification and/or cleaning, sterilization, disinfection, film deposition, and etching of discrete objects, powder-like substances and continuous media by generating a stimulated light emission in the chamber wherein treated surfaces are placed. The apparatus may operate by itself or together with plasma chamber and/or means for adding an active monomer vapor.

### BACKGROUND OF THE INVENTION

1. Field of the invention

Present invention relates in general to apparatuses and methods of materials processing. More particularly, present invention relates to apparatuses and methods in which materials processing including surface modification and/or cleaning, sterilization, disinfection, film deposition, and etching is accomplished or enhanced by light irradiation specifically by stimulated light irradiation.

2. Description of the Prior Art

Materials processing is a part of manufacturing. It comprises numerous groups of methods including those related to surface modification and/or cleaning, sterilization, disinfection, film deposition, and etching. Said methods are applied to semiconductor chips, plastic devices, metallic items, etc.

Surface modification improves wettability, printability, dyeability, adhesion, water repellency, oil repellency, biocompatibility of treated surfaces while leaving intact such characteristics of bulk material as strength, density, heat capacity, electrical resistivity, etc.

Cleaning is a non-selective removal of unwanted materials such as dust, dirt, grease, various oils, etc. in preparation for usage or for further processing.

Sterilization and disinfection involve the destruction of harmful living microorganisms such as pathogenic microbes and viruses, spores, fungi, etc.

Deposition includes creation of thin and thick film coatings. Thin film coatings are used for example in semiconductor chips or biomedical devices. Thick film coatings are used for example as reflective layers on lenses, protective films for magnetic disks, resists for photolithography.

Etching is widely used commercially for selectively removing unwanted materials, e.g. in such semiconductor chip manufacturing processes as silicon wafer photoresist stripping or removing drill smears in print circuit boards.

Apparatuses and methods of materials processing using stimulated light irradiation produced by laser beams are well known in the art. Typically such processing takes place in a vacuum chamber wherein target and treated objects are installed. Laser beam hits the target and evaporates its material by ablation. The species comprising plume of evaporated material deposit on surfaces of treated objects.

Such a method is described, e.g. in U.S. Pat. No. 4,604,294 “Process for forming an organic thin film” issued to M. Tanaka, et al. Laser beam passing through the vacuum chamber's window hits a target, material is evaporated from the target and deposits on a substrate. Using said method, the thickness of deposited thin film is not uniform across the substrate's surface and film's stoichiometry may differ from that of the target material. The latter means chemical structure of deposited thin film is not identical to that of the target material, i.e. whatever is deposited is not exactly what desired to be deposited. Moreover, said method incurs energy losses due to laser beam passing through partially reflective mirror, air and the vacuum chamber's window.

In U.S. Pat. No. 5,015,495 “Method and apparatus for pulsed induced vapor deposition of thin films” issued to J. Venkatesan and X. Wu the laser beam intensity is selected so high that all constituents of target material would vaporize. Such approach provides vapor stoichiometry closer to that of the target material. However, high intensity laser light may also break chemical bonds in vaporized molecules reducing aforementioned effect of better stoichiometry reproduction. Also, problems with uniformity of deposited film thickness and energy losses remain unresolved.

Apparatuses that utilize vapor produced by laser ablation require scanning of target by laser beam because area of ablation is very small. In U.S. Pat. No. 5,534,489 “Method of fabricating oxide superconducting films by laser deposition” issued to N. Hayashi and N. Yoshida the area of ablation is increased by letting laser beam through a cylindrical lens. Such an approach improves deposited film thickness uniformity. However, problems of inadequate stoichiometry and laser light energy losses persist in this method too.

In U.S. Pat. No. 5,820,682 “Laser deposition apparatus for depositing a large area oxide thin films on a substrate” issued to G. Sung and J. Suh laser light energy losses are reduced by placing laser inside a vacuum chamber. The apparatus is rather complex involving as it is rotation of treated object in vacuum. Moreover, it does not resolve problem of different stoichiometry in targeted and deposited materials.

One may conclude that laser deposition apparatuses and methods known to the art do not resolve main problems of economically providing high quality uniform films on surfaces of treated objects.

Laser etching is accomplished by beaming laser light onto a treated surface. Material is removed from said surface by ablation. An apparatus for laser etching is described, e.g. in U.S. Pat. No. 6,123,803 “Laser processing chamber with cassette cell” issued to M. Genut, et al. Problem with laser etching methods is that they require scanning of treated surfaces by moving either laser beam or treated objects.

By far, the most commercially used method of surface modification, materials deposition and dry etching is plasma treatment. Plasma consists of neutral atoms and molecules either in ground state or in excited states, chemical radicals, ions, and electrons. Overall, the number of positive species in plasma is equal to the number of negative ones. So plasma as a whole is neutral. However, locally concentration of charged species might exceed the average value resulting in non-zero electrical potential. This potential speeds up chemical reactions. Thus reactions that normally occur only at elevated temperatures proceed at room temperature in plasma. This feature of plasma provides an enormous advantage in many commercially important processes because required results could be achieved without damaging treated materials by exposure to high temperatures.

Plasma treatment could be direct or remote. Direct plasma utilizes deep ultraviolet light and highly energetic species like ions, electrons, excited atoms, molecules and chemical radicals. Said species and photons of ultraviolet light activate a great variety of chemical reactions. Only a small part of said reactions is beneficial to materials processing while some of them are even harmful causing damage to the treated surfaces. Remote plasma is milder than the direct one using less energetic species and having no ultraviolet photons. However, this is achieved at the expense of more complex apparatuses. Also, lack of ultraviolet light sometimes is not beneficial to materials processing.

In U.S. Pat. No. 4,820,377 “Method for cleanup processing and vacuum process module” issued to Davis, et al. chemical reactions are enhanced by additional light sources, radiant heaters and plasma generated ultraviolet light. Radiant heaters do not have enough energy to break chemical bonds. They may only heat treated surfaces up promoting annealing. By contrast, deep ultraviolet radiation from plasma has sufficient energy to break chemical bonds. However, plasma generated ultraviolet light in this apparatus is useful only in the direct plasma—remote plasma combinations because remote plasma is lacking ultraviolet light and said ultraviolet light is supplied by direct plasma source placed close to the remote plasma source and separated from said remote plasma source by media transparent to ultraviolet light generated by direct plasma. Realization of said combination of direct and remote plasmas requires a rather complex apparatus. Also, pressure range in which such combination is operable is limited because at pressures higher than about a torr plasma does not ignite.

Chemical reactions in plasma might be enhanced by applying additional energy to gas atoms and molecules and to the molecules of treated surfaces in the form of light irradiation including laser light irradiation. In U.S. Pat. No. 4,664,769 “Photoelectric enhanced plasma glow discharge system and method including radiation means” issued to J. Cuomo and C. Guarnieri chemical reactions in plasma are enhanced by means of ultraviolet laser beaming through the window which is capable of transmitting ultraviolet radiation. This method requires scanning of treated objects by laser beam and useful energy is lost due to passing of laser light through partially reflective mirror, ambient atmosphere, and window.

### BRIEF SUMMARY OF THE INVENTION

The present invention provides an apparatus and a method for materials processing. It is especially suitable for materials surface modification and/or cleaning, sterilization, disinfection, film deposition, and etching. Materials processing using apparatus and method of the present invention could be a part of manufacturing of various plastic devices, semiconductor electronics circuitry, metallic items, etc. Said processing may apply to discrete objects like catheters, silicon wafers, contact lenses, etc., to powder-like substances like granules, beads, powders, pellets, etc., and to continuous media like films, webs, cables, wires, etc.

The objective of the present invention is to provide with highly effective and selective means of materials processing and a method thereof by using stimulated light emission generated by gases capable of population inversion.

Another objective is to use fluidization of powder-like substances to intensify their processing by stimulated light emission.

Another objective is to use rotation of continuous media while exposing its surfaces to stimulated light emission.

Yet another objective is to enhance stimulated light emission treatment with chemical radicals from remote plasma source.

Yet another objective is to enhance stimulated light emission treatment by a follow-up with highly reactive monomer vapor that attaches to the chemical radicals created by stimulated light emission on the treated surfaces.

The apparatus of the present invention consists of the treatment chamber connected with pumps, gas containers, and a power generator. The treatment chamber comprises means for producing population inversion either in a gas moving through said chamber or in a gas filling an enclosure with window transparent to stimulated photons. Said means may include a pair of electrodes connected to a power generator and to a pulse generator. Also, said means may include an optical resonator. It is essential for the present invention that said optical resonator is unstable and if said resonator is formed by concave mirrors said mirrors are fully reflective.

If treated objects are discrete, then they are placed on an object holder which typically is a perforated plate its perforations allowing free passage of gas or gaseous mixture circulated by a pump or moved by a vacuum pump.

If treated objects are powder-like substances, they also could be placed onto a perforated plate with such sizes of perforations that at a given pressure drop the fluidization of powder particles is achieved. Also, special inserts could be installed in said perforations to prevent the backward seepage of fluidized particles.

If a treated object is continuous media in a roll form, the treatment chamber comprises two shafts upon which cores are installed for take-off and take-on of said media and at least one motor is connected to a shaft to enable the unwinding of said media.

Materials processing according to the present invention is performed by moving gas or gaseous mixture capable of producing species useful for surface modification and/or cleaning, sterilization, disinfection, film deposition, and etching of treated objects through the treatment chamber wherein stimulated light emission is generated. Said emission in its turn promotes chemical reactions that modify treated surfaces.

### DESCRIPTION OF THE DRAWINGS

Herebelow an apparatus for stimulated light emission treatment according to the present invention and a practical method thereof are described in several embodiments with reference to the drawings. Like numerals refer to like parts.

These drawings are for illustration purposes only. They are not to scale and presented only to illustrate concepts of the present invention.

FIG. 1 is a schematic diagram of the apparatus for stimulated light emission treatment of discrete objects.

FIG. 2 shows a frontal cross-sectional view of the stimulated light emission treatment chamber.

FIG. 3 shows a cross-sectional view of the stimulated light emission treatment chamber from the bottom.

FIG. 4 is a schematic diagram of electrical circuitry for the stimulated light emission treatment chamber.

FIG. 5 shows a ray pattern diagram in an unstable optical resonator formed by concave mirrors.

FIG. 6 is a schematic diagram of optical resonator with a non-linear crystal.

FIG. 7 is a schematic diagram of optical resonator with a target plate.

FIG. 8 is a schematic diagram of electrical circuitry for the stimulated light emission treatment chamber with additional direct plasma electrical circuit.

FIG. 9 is a schematic diagram of electrical circuitry for conventional plasma chamber.

FIG. 10 is a schematic diagram of the apparatus for stimulated light emission treatment of discrete objects with enclosure that comprises optical resonator, reflector and electrodes.

FIG. 11 shows a frontal cross-sectional view of the stimulated light emission treatment chamber with electrodes and mirrors placed within an enclosure.

FIG. 12 is a schematic diagram of the apparatus for stimulated light emission treatment of powder-like substances.

FIG. 13 shows a frontal cross-sectional view of the chamber for stimulated light emission treatment of powder-like substances.

FIG. 14 is a schematic diagram of the apparatus for stimulated light emission treatment of powder-like substances with electrodes and mirrors placed within an enclosure.

FIG. 15 shows a frontal cross-sectional view of the chamber for stimulated light emission treatment of powder-like substances with electrodes and mirrors placed within an enclosure.

FIG. 16 is a schematic diagram of the apparatus for stimulated light emission treatment of continuous media.

FIG. 17 shows a frontal cross-sectional view of the apparatus for stimulated light emission treatment of continuous media.

FIG. 18 shows a bottom view of the apparatus for stimulated light emission treatment of continuous media.

FIG. 19 is a schematic diagram of the apparatus for stimulated light emission treatment of continuous media with electrodes and mirrors placed within an enclosure.

FIG. 20 shows a frontal cross-sectional view of the apparatus for stimulated light emission treatment of continuous media with electrodes and mirrors placed within an enclosure.

FIG. 21 shows a bottom view of the apparatus for stimulated light emission treatment of continuous media with electrodes and mirrors placed within an enclosure.

FIG. 22 shows a frontal cross-sectional view of the apparatus for stimulated light emission treatment of continuous media with precoating.

FIG. 23 shows a bottom view of the apparatus for stimulated light emission treatment of continuous media with precoating.

### DETAILED DESCRIPTION OF THE INVENTION

The method of the present invention applies to the following: 1) discrete objects, e.g. silicon wafers, liquid crystal displays, ink jet printer heads, plastic catheters, vascular prostheses, biosensors, heart valves, dental implants, orthopedic implants, contact lenses, solar cells, non-stick frying pans, etc.; 2) powder-like substances, i.e. granules, beads, pellets, powders e.g. ion-exchange beads, pills, buckminster fullerene nanopowders, etc.; 3) continuous media, i.e. films, cables, wires, woven or non-woven materials in roll form, e.g. plastic bag materials, fabrics, web membranes, optical fibers, etc.

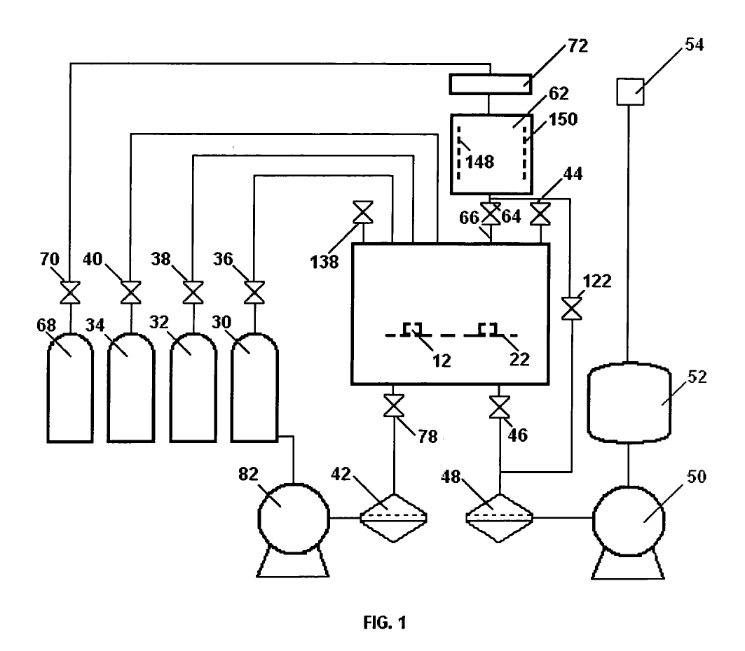

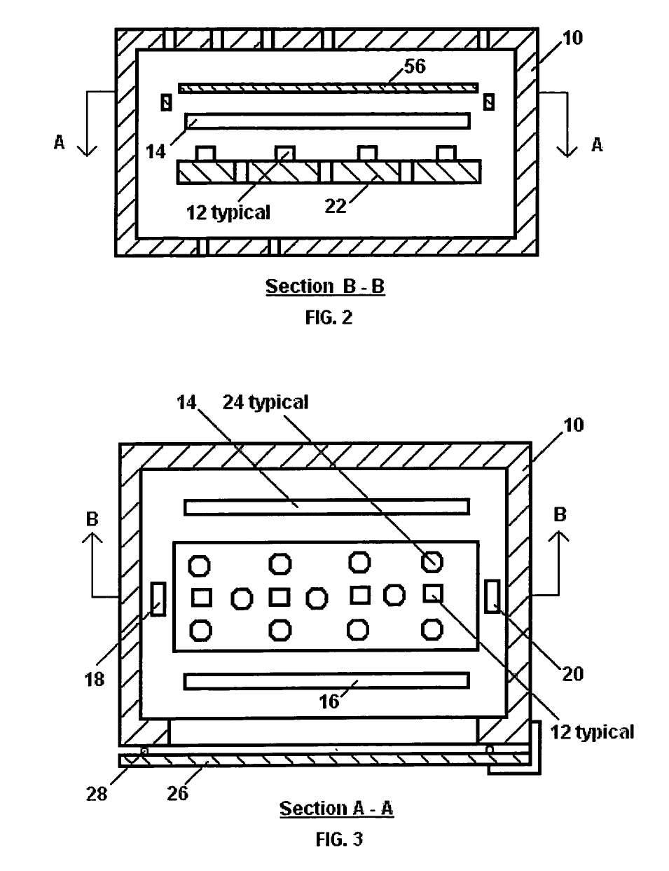

Schematic diagram of the apparatus realizing the method of the present invention for discrete objects is shown in FIG. 1. Frontal cross-section of the treatment chamber 10 and the cross-section viewed from the bottom of said chamber are shown in FIG. 2 and FIG. 3 respectively.

Processing comprises the following steps: 1) loading objects 12 to be treated into chamber 10; 2) evacuation of chamber 10; 3) treatment of objects 12 by stimulated light emission; 4) evacuation of chamber 10 after said treatment; 5) purging chamber 10; 6) breaking vacuum in chamber 10; 7) unloading treated objects 12.

The processing starts with loading discrete objects 12 into chamber 10 onto support means 22, e.g. onto a perforated plate. Objects 12 should be placed as uniformly as possible between perforations 24. The purpose of said perforations is to provide free passage of gas around objects 12. After objects 12 are loaded, door 26 is closed and sealed by any suitable means, e.g. by O-ring 28.

After door 26 is closed and sealed, vacuum pump 50 is turned on, valve 46 is open, and evacuation of chamber 10 begins. Any suitable vacuum pump could be used for evacuation, for example a mechanical rotary vane pump made by any vacuum equipment manufacturer, e.g. by BOC Edwards, Leybold Vacuum Products, Stokes Vacuum, etc. and could be acquired through any major distributor of vacuum equipment such as Kurt J. Lesker Company.

Evacuation is needed to remove air from chamber 10 before filling it with gas or gaseous mixture from container 30. After evacuation is completed, pump 50 is turned off, valve 46 is closed, pump 82 is turned on, valves 36 and 78 are open, and gas or gaseous mixture from container 30 starts circulating through chamber 10.

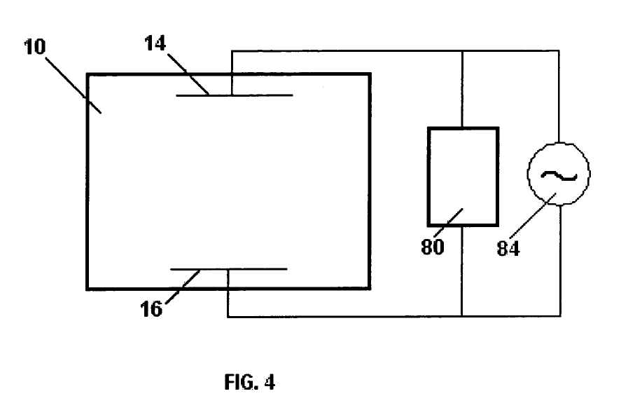

Said gas or gaseous mixture is activated by electrical discharges between electrodes 14 and 16. Electrical schematics of the apparatus of the present invention is shown in FIG. 4. Electrodes 14 and 16 are connected to high voltage power supply 84 and if necessary to pulse generator 80. Said power supply converts standard alternating current to high voltage. Said high voltage should be sufficient to ensure presence of electrical current in said gas. High voltage power supply 84 and pulse generator 80 for that matter are standard laser components and could be acquired through a distributor like Cascade Laser Corp. or from established manufacturers such as Power Technology, Inc., Optelectra, etc.

Gas or gaseous mixture flowing from container 30 through chamber 10 should be capable of undergoing a population inversion during electrical discharges between electrodes 14 and 16. Population inversion is a state of gas. In said state more atoms or molecules comprising said gas are at metastable upper energy level than at lower energy level. Typically, metastable energy level is a level at which an atom or molecule does not deexcite back to a lower level for a time interval of about a hundred thousand times longer than the normal deexcitation time of about 10 nanoseconds.

Said atoms or molecules may absorb or emit energy in the form of photons. Energy emission could be either spontaneous or stimulated. Spontaneous emission happens at random and emitted photons may have any wavelength. By contrast, stimulated emission happens only when any of said atoms or molecules is approached by a photon with the energy equal to the difference between higher metastable and lower energy levels. After being approached by said photon the atom or molecule emits another photon with the same energy and phase moving in the same direction as the first one.

Gases that can produce population inversion include but are not limited to argon, neon, krypton, xenon, helium, nitrogen, carbon dioxide, carbon monoxide, fluorine, chlorine, iodine, bromine, hydrogen and gaseous mixtures thereof. They can be acquired through established compressed gas distributors, e.g. through Air Products and Chemicals, Matheson, etc. In a distributor catalog they are typically referred to as laser gases and include pure gases as well as off-the-shelf and custom-manufactured gaseous mixtures.

Said gases are characterized by optical gain they are capable to produce. An optical gain is a measure of how well gas molecules amplify photons by stimulated light emission. When optical gain is relatively small an optical resonator is required. Some gases possess such high optical gain they do not require an optical resonator. An example of gas that does not require an optical resonator is nitrogen. The selection of gas depends on the application because different applications require different gases to accomplish.

If treated material efficiently absorbs light at the stimulated light emission wavelength said material is treated per se without any additions. If however light absorption is not sufficient for desired reactions to take place at the treated surfaces it is preferable to add small quantities of photoinitiators to the treated material. Examples of suitable photoinitiators include but are not limited to benzophenones, xanthones, thioxanthones, fluorenones, polycyclic quinones, O-acylated oximinoketones, alkoxy phenyl ketones.

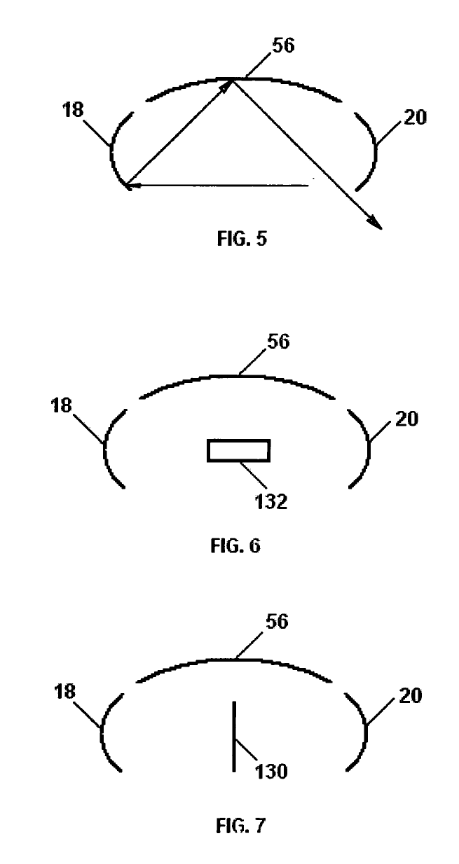

Schematic diagram of the unstable optical resonator formed by mirrors 18 and 20 is shown in FIG. 5. Cover 56 serves as a reflector preventing stimulated photons to escape to the walls of chamber 10 and directing said photons onto treated objects 12 instead. Arrows indicate a path of a stimulated photon. Said photon bounces back and forth between fully reflective mirrors 18 and 20. After leaving the resonator a stimulated photon either moves in the direction of holder 22 where treated objects 12 are located or in the opposite direction. In the latter case it is reflected by cover 56 in the direction of holder 22. Said optical resonator differs from that of a gas laser in five important aspects.

First, all mirrors comprising said optical resonator are fully reflective while in gas laser one of the mirrors are partially reflective. The reason is that in the apparatus of the present invention stimulated photons stay in chamber 10 and therefore there is no need for them to exit through a partially reflective mirror like they do in a laser.

Second, the resonator of the present invention is unstable while a gas laser typically employs a stable resonator. In an unstable resonator a ray of light leaves said resonator after a certain number of reflections. An example of unstable optical resonator is a pair of concave mirrors with parameters that satisfy certain inequalities. Said inequalities depend on distance between mirrors and their radii of curvature. They can be found in the monograph “Lasers. Theory and Practice” by John Hawkins and Ian Latimer, Prentice Hall, 1994. The parameters of said unstable optical resonator must be selected in such a way that enough photons are produced in the space between said mirrors to sustain stimulated light emission while simultaneously as many stimulated photons as possible are redirected to treated objects 12.

Third, the resonator of the present invention has a fully reflective mirror 56 prohibiting stimulated photons from moving in the direction opposite the location of treated objects 12. A laser resonator does not need such a reflector.

Fourth, inside a gas laser optical resonator the number of trips traveled by a stimulated photon determines only laser's power, the larger the number of trips the better, while in the apparatus of the present invention the resonator, in addition to providing required power, should also be designed in such a way that some of the trips result in interactions of stimulated photons with gas atoms or molecules. Said interactions promote chemical reactions that produce useful chemical functional groups.

Fifth, inside a gas laser optical resonator the gas pressure should be suffcient only to provide for population inversion while in the apparatus of the present invention the gas pressure in addition to ensuring population inversion should also provide for migration of useful chemical functional groups produced by interactions of stimulated photons with gas atoms and/or molecules to the surfaces of treated objects 12 during residence time inside chamber 10 of said gas atoms and/or molecules.

Mirror material could be aluminum, silver, gold, or any other suitable metal. Properly configured and polished parts of the walls could serve as mirrors. Typically fully reflective metallic mirrors reflect about 95% of photons that impinge on their surfaces. The rest is absorbed and converted to heat. Accumulation of heat may damage a mirror. Therefore cross-sections of means connecting mirrors to the walls of chamber 10 should be sufficiently large to conduct heat generated in mirrors to the walls. If heat accumulation in mirrors prevails over thermal conduction through said means, metallic mirrors could be replaced by multiple layers of dielectric. Said multiple dielectric mirrors experience much less light absorption than the metallic ones.

To further enhance performance of said optical resonator a non-linear crystal 132 (see FIG. 6) could be installed between mirrors 18 and 20. Material of said crystal is selected in such a way as to reduce the wavelength of a stimulated photon. The smaller the wavelength the larger the photon's energy. An example of such material is lithium iodate LilO₃. Said materials could be acquired through commercial sources, e.g. from Photox Optical Systems, Castech, etc.

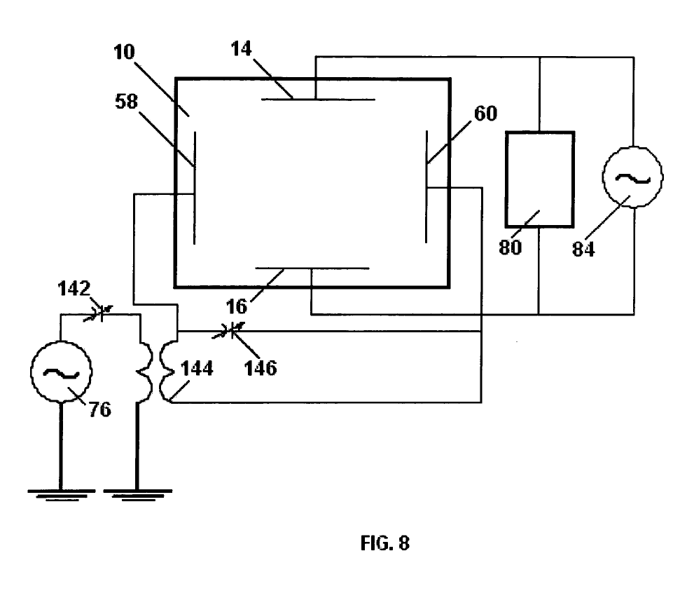

A plate 130 or any other means covered with material that volatilizes under irradiation of stimulated photons could be installed inside chamber 10 (see FIG. 7). After volatilization, molecules of said material may drift to treated objects 12 and deposit on surfaces of said objects. Positioning of target plate 130 under fully reflective mirrors 18, 20 and 56 provides a certain advantage over conventional approach of target coating ablation by a laser beam because said positioning allows to eliminate energy losses due to passing of a beam through a partially reflective mirror.

After processing of objects 12 has been completed, valve 36 is closed and pump 82 evacuates contents of chamber 10 into container 30. After said evacuation is completed, valve 78 is closed, pump 82 is turned off, vacuum pump 50 is turned on, and valve 46 is open. After final evacuation of chamber 10, valve 38 is open for a short period of time, e.g. for a few seconds, and chamber 10 is purged by an inert gas, e.g. nitrogen from container 32. After purging, valves 38 and 46 are closed, vacuum pump 50 is turned off, and vacuum is broken by opening vent valve 44. Then door 26 is open and treated objects 12 are unloaded.

An improvement of bondability of spacecraft polypropylene parts can serve as an example of the method described above. Polypropylene is inherently hydrophobic and when parts are being bonded by glue said glue does not fill certain surface rough spots reducing effectiveness of bonding thereby.

Polypropylene may comprise a photoinitiator, e.g. from 0.1% to 4% by weight of 4-methyl-benzophenone commercially available from Sartomer Company under the trade name of EsaCure.

After parts 12 are loaded into chamber 10 and evacuation of said chamber is completed, pump 82 is turned on, valves 36 and 78 are open, and gaseous mixture from container 30 starts circulating through chamber 10. Said gaseous mixture comprises argon 9% by volume, tetrafluoromethane 20% by volume, oxygen 1% by volume, helium the rest.

When said gaseous mixture flows through chamber 10 it is subjected to electrical discharges between electrodes 14 and 16 in the range between ten and thirty kilovolt. Said discharges are delivered in pulses. Each pulse is twenty to forty nanosecond long. The frequency of pulses is from ten hertz to two kilohertz.

Under high voltage discharge a portion of tetrafluoromethane decomposes into carbon and fluorine radicals. Some of said fluorine radicals combine with argon forming argon fluoride dimers. Said dimers exist only when they are excited ensuring the population inversion during discharge. Each time pulse generator 80 interrupts the discharge they decompose producing photons of stimulated light emission with wavelength of 193 nanometers.

The energy Eₚ of photon is proportional to its frequency ν namely Eₚ=hν where h=6.6252*10⁻³⁴ Joule*second is the Planck's constant. Frequency ν=c/λ where c=2.9979*10⁸ meter/second is the speed of light and λ is the wavelength of photon. So the energy of photon Eₚ=hc/λ. The energy of mole of photons is E=AEₚ=Ahc/λ where A=6.0225*10²³ is the Avogadro's constant. Therefore, a mole of stimulated photons with wavelength of 193 nanometers possesses energy E=Ahc/λ=6.0225*10²³*6.6252*10⁻³⁴*2.9979*10⁸/(193*10⁻⁹)=620*10³ joule/mole=620 kilojoule/mole. As said energy is larger than the energy required to break bonds in oxygen molecule which is 494 kilojoule/mole, oxygen molecules dissociate into oxygen atoms upon collision with stimulated photons.

Because the resonator formed by mirrors 18 and 20 is unstable said stimulated photons eventually leave it. Some of the photons that reach objects 12 are absorbed at the treated surfaces. The energy of absorbed photons with wavelength of 193 nanometers is equal to 620 kilojoule/mole. The energy necessary to break a hydrogen-carbon bond is 415 kilojoule/mole. As the energy required to break a hydrogen-carbon bond is less than the energy of photon with the wavelength of 193 nanometers, said bonds are broken upon collisions with stimulated photons and chemical radicals are generated at scission sites at or very close to the treated surface. Oxygen atoms created by dissociation of oxygen molecules by stimulated photons drift to the treated surfaces wherein they react with said radicals forming oxygen functional groups such as hydroxyl, carboxyl, and carbonyl groups.

The gas pressure governed by openings of valves 36 and 78 is selected so as to provide with enough gas molecules for efficient generation of stimulated photons and to have as less molecules as possible to ensure fast drifting of oxygen atoms to the treated surfaces.

If benzophenone is added to a polymer, energy of stimulated photons is sufficient to excite benzophenone molecule to the singlet state. A part of benzophenone molecules in the singlet state cross without radiation to the excited triplet state. The benzophenone in the triplet state abstracts hydrogen from the polymer backbone generating chemical radicals there. Said radicals react with oxygen atoms created by dissociation of oxygen molecules during collisions with stimulated photons. As a result of said reactions oxygen functional groups are created, e.g. hydroxyl, carboxyl, and carbonyl groups.

If a non-linear crystal 132 is installed between mirrors 18 and 20 there is a mix of stimulated photons some with the energy of 620 kilojoule/mole and others with larger energy, e.g. with the energy of 1,240 kilojoule/mole. Though the presence of non-linear crystal 132 slightly reduces the total amount of stimulated photons due mainly to the raise of crystal's temperature during operation said reduction is more than compensated by the increase in the amount of more effective high power photons.

After a certain number of pulses for example after 30,000 pulses power supply 84 and pulse generator 80 are turned off, valve 36 is closed, and pump 82 continues to pull contents of chamber 10 back to container 30. Then pump 82 is turned off, vacuum pump 50 is turned on, valve 46 is open, and chamber 10 is fully evacuated. Then valve 38 is open and chamber 10 is purged by nitrogen from container 32. After purging, vacuum is broken by opening vent valve 44. Then door 26 is open and treated parts 12 are unloaded.

The bondability of surfaces of treated polypropylene parts 12 is greatly enhanced because oxygen functional groups generated both by stimulated photons proper and through transformed photoiniators significantly increase surface energy. A glue smoothly flows over the treated surfaces filling all cracks and crevices there.

If gas from container 30 is capable of achieving a population inversion in vacuum, the circulation by pump 82 could be replaced with exhausting said gas into the atmosphere by vacuum pump 50. Said exhausting does not adversely affect the environment because gas quantities are minute and there is a scrubber 52 that converts any hazardous species into those not harmful to the environment.

Processing of discrete objects without gas circulation comprises the same seven steps as the processing with gas circulation described above. However, after evacuation of chamber 10 is completed and the stimulated light emission treatment is about to begin, vacuum pump 50 continues to be open with valves 36 and 46 open and valve 78 closed letting gas from container 30 to flow through chamber 10 into the atmosphere via filter 48, vacuum pump 50, scrubber 52, and vent 54.

In chamber 10 flowing gas is activated by electrical discharges between electrodes 14 and 16. Because said gas is capable of achieving population inversion in vacuum, stimulated photons are created during said electrical discharges. Said stimulated photons promote treatment of objects 12. After said treatment is completed, chamber 10 is evacuated, purged and vacuum is broken by opening vent valve 44. After breaking vacuum and opening door 26, treated objects 12 are unloaded.

An example of stimulated light emission treatment without gas circulation is sterilization of plastic blood bags 12. The gas selected for treatment of said bags is nitrogen. It fills container 30.

The processing starts with loading said bags onto holder 22 between perforations 24. Electrodes 14 and 16 are situated as close to object holder 22 as possible so as to have said bags located between said electrodes. After loading, door 26 is closed and sealed, vacuum pump 50 is turned on, valve 46 is open and chamber 10 is evacuated. After evacuation of chamber 10 is completed, valve 36 is open letting nitrogen from container 30 into chamber 10. Valve 46 remains open to maintain a certain flow rate through chamber 10. At said flow rate the pressure inside chamber 10 is in the range between 80 torr and 120 torr.

If the apparatus is located in a clean room, clean air could be used instead of nitrogen. In such a case, after evacuation of chamber 10 is completed, valve 138 is open letting air from the clean room into chamber 10. Valve 46 remains open to maintain the partial pressure of nitrogen inside chamber 10 within the pressure range between 80 torr and 120 torr.

Simultaneously with the start of the nitrogen flow power supply 84 and pulse generator 80 are turned on. Voltage is in the range of ten kilovolt to twenty kilovolt. Pulse generator 80 maintains duration of an electrical discharge between 2 nanosecond and 12 nanosecond and frequency of discharges between 50 hertz and 200 hertz.

During each pulse nitrogen molecules achieve population inversion and emit stimulated ultraviolet light with the wavelength of 337.1 nanometers. As opposed to laser that uses only a part of stimulated photons namely those moving through a partially reflective mirror, present invention uses all stimulated photons moving in every direction. Moreover, during a pulse each nitrogen molecule located between electrodes 14 and 16 is a potential source of ultraviolet light. Because said molecules are uniformly distributed, present invention has a significant advantage over conventional ultraviolet sterilizing systems where only those parts of surfaces that are in the line of sight of ultraviolet source are treated and shaded parts remain untreated.

To complement the stimulated light emission, chamber 10 may comprise a plurality of conventional ultraviolet lamps (not shown).

After the stimulated light emission treatment is completed, valves 36 and 46 are closed, vacuum pump 50 is turned off, and vacuum is broken by opening vent valve 44. After pressure inside chamber 10 reaches the ambient value, door 26 is open and sterilized blood bags 12 are unloaded.

Another example of stimulated light emission treatment without gas circulation is etching of silicon wafers in the fabrication of ultralarge—scale integrated circuits on semiconductor chips. At a certain step of said fabrication a silicon wafer is covered with photoresist mask. Areas unprotected by said mask are etched to produce trenches wherein a useful material, e.g. a metal is subsequently deposited.

Container 30 is filled with gaseous mixture of argon 20% by volume, chlorine 20% by volume, hydrogen the rest. After silicon wafers 12 covered with photoresist are loaded onto perforated plate 22 by installing said wafers into any suitable holding means, e.g. into cassettes (not shown), door 26 is closed and sealed, vacuum pump 50 is turned on, valve 46 is open. Then chamber 10 is evacuated. After evacuation, valve 36 is open and said gaseous mixture is pulled by vacuum pump 50 through chamber 10, filter 48, scrubber 52, and vent 54 into the atmosphere. Valve 36 should be open in such a way as to allow maintaining the partial pressure of argon inside chamber 10 at about 300 millitorr.

Simultaneously power supply 84 is turned on starting electrical discharges between electrodes 14 and 16 at 100 volt and current of about 5 ampere. Said electrical discharges stimulate argon ions to produce photons with a few wavelengths in the range between 457.9 nanometers and 514.5 nanometers.

It is taught in the article “Laser Studies of Surface Chemistry” by G. Selwyn and M. Lin published in the book “Lasers as Reactants and Probes in Chemistry” edited by W. Jackson and A. Harvey, Howard University Press, Washington, D.C., 1985 that stimulated photons with the wavelengths in said range dissociate chlorine molecules into chlorine atoms. Said chlorine atoms etch areas of silicon wafers unprotected by photoresist.

After etching has reached the predetermined depth, valve 36 is closed and chamber 10 is evacuated by vacuum pump 50, purged by nitrogen from tank 32, and vacuum is broken by opening vent valve 44. Then door 26 is open and etched wafers 12 are unloaded.

Processing per method of the present invention could be enhanced by using monomer vapor. Examples of said monomers are allyl amine (vapor pressure of 187 torr at 20° C.), vinyl acetate (vapor pressure of 100 torr at 20° C.), allyl alcohol (vapor pressure of 23 torr at 20° C.), styrene (vapor pressure of 9 torr at 20° C.), acrylic acid (vapor pressure of 2.85 torr at 20° C.), cyclooctamethylsiloxane (vapor pressure of 1.5 torr at 20° C.), methacrylic acid (vapor pressure of 1 torr at 20° C.), glycidyl methacrylate (vapor pressure of 330 millitorr at 20° C.), hydroxypropyl acrylate (vapor pressure of 160 millitorr at 20° C.), N-vinyl pyrrolidone (vapor pressure of 90 millitorr at 20° C.), hydroxyethyl methacrylate (vapor pressure of 75 millitorr at 20° C.), acrylamide (vapor pressure of 7 millitorr at 20° C.). In addition to chemical functional groups created by stimulated photons, monomer reacts with radicals created on the treated surfaces by stimulated emission and polymerizes on said surfaces enhancing the effect of the treatment thereby.

Said monomer vapor could be added to the gaseous mixture filling container 30.

If this is the case said monomer reacts with the treated surface during stimulated light emission treatment step described above.

Also, monomer vapor could be added after stimulated light emission step is completed. If this is the case valve 36 is closed and chamber 10 is evacuated after completion of the stimulated light emission step until pressure inside chamber 10 becomes less than vapor pressure of monomer kept in container 34. Then valve 40 is open and monomer vapor starts flowing into chamber 10 from container 34. Molecules of monomer react with radicals created by stimulated light emission on the treated surfaces and polymerize on said surfaces.

An example of said treatment is surface modification of polyethylene catheters. Polyethylene is inherently hydrophobic. If the catheter's surface is left hydrophobic it may carry tiny air bubbles into blood stream upon insertion of a catheter into a blood vessel. Inside capillaries said bubbles may become emboli and block the blood flow.

The gaseous mixture placed in container 30 is argon 9% by volume, tetrafluoromethane 20% by volume, oxygen 1% by volume, helium the rest. Said gaseous mixture is kept in container 30 under pressure of 1 to 10 megapascal.

At the start of the processing catheters 12 are loaded onto holder 22 between perforations 24. After loading is completed, door 26 is closed and sealed by Viton O-ring 28. Then valve 46 is open and vacuum pump 50 starts evacuation of chamber 10.

After evacuation is completed, vacuum pump 50 is turned off, valve 46 is closed, valves 36 and 78 are open, pump 82 is turned on starting circulation of gaseous mixture through chamber 10.

Said gaseous mixture is subjected to electrical discharges between electrodes 14 and 16 in the range between ten kilovolt and thirty kilovolt. Said discharges are delivered in pulses. Each pulse is 20 nanosecond to 40 nanosecond long. The frequency of pulses is from 10 hertz to 2 kilohertz.

Under high voltage discharges a portion of tetrafluoromethane decomposes into carbon and fluorine radicals. Said fluorine radicals combine with argon forming argon fluoride dimers. Said argon fluoride dimers produce photons of stimulated light emission at the wavelength of 193 nanometers. Said stimulated photons move back and forth bouncing off mirrors 18 and 20. Upon collisions with oxygen molecules they cause them to dissociate into oxygen atoms.

Because resonator formed by mirrors 18 and 20 is unstable said stimulated photons eventually leave it. Some of them hit treated surfaces of polyethylene catheters and are absorbed there. Energy of absorbed photons is enough to break bonds in polyethylene backbone producing radicals at or very close to the treated surface. Said radicals react with said oxygen atoms forming oxygen functional groups such as hydroxyl, carboxyl, and carbonyl groups on the surfaces of treated catheters.

If benzophenone is added to polyethylene, energy of stimulated photons is sufficient to excite benzophenone molecule to the singlet state. Some benzophenone molecules in the singlet state cross without radiation to the excited triplet state. The benzophenone in the triplet state abstracts hydrogen from the polyethylene backbone generating a radical there. Said radicals react with said oxygen atoms forming oxygen functional groups such as hydroxyl, carboxyl, and carbonyl groups.

If a non-linear crystal 132 is installed between mirrors 18 and 20 there is a mix of stimulated photons, some with the energy of 620 kilojoule/mole and others with the energy of 1,240 kilojoule/mole. Stimulated photons with higher energy are more likely to produce useful oxygen functional groups on the surfaces of catheters.

Said oxygen functional groups generated by both stimulated photons and through transformed photoiniators significantly increase surface energy enhancing its wettability thereby.

After a certain number of pulses for example after 30,000 pulses power supply 84 and pulse generator 80 are turned off, valve 36 is closed, and pump 82 evacuates chamber 10. Then valve 78 is closed, pump 82 is turned off, vacuum pump 50 is turned on, valve 46 is open, and chamber 10 is evacuated until pressure of gaseous mixture becomes less than 75 millitorr at 20° C., the vapor pressure of hydroxyethyl methacrylate, a hydrophilic monomer kept in container 34. When pressure in chamber 10 is below said vapor pressure, valve 40 is open letting vapor of hydroxyethyl methacrylate from container 34 into chamber 10. Molecules of said monomer attach to radicals at the treated surfaces enhancing wettability even further. Duration of monomer treatment is from a few seconds to a few minutes depending on how many radicals had been generated.

After monomer deposition step has been completed, chamber 10 is evacuated and then purged by opening valve 38 and letting nitrogen from tank 32 in. After purging, vacuum is broken by opening vent valve 44. After pressure inside chamber 10 becomes equal to atmospheric, vacuum pump 50 is turned off, valve 46 is closed, door 26 is open, and treated catheters are unloaded. It is beneficial to place unloaded catheters into hydrogel bath, e.g. into polyvinyl alcohol hydrogel bath. Hydrogel coating enhances lubricity of catheter while the latter moves inside a blood vessel.

Processing per method of the present invention may be enhanced also by combining the stimulated light emission treatment step with plasma treatment. Said additional plasma treatment could be either direct or remote. Direct plasma treatment utilizes all species generated by plasma glow namely excited atoms and molecules, ions, electrons, and chemical radicals. Therefore, it is not very selective. By contrast, remote plasma treatment uses only chemical radicals which is advantageous in the applications where ion bombardment is not necessary or even deleterious.

Additional direct plasma could be generated before, after or during stimulated light emission step by turning on a power supply 76 connected via matching network that comprises capacitors 142 and 146 and transformer 144 to either a pair of electrodes 58 and 60 (see FIG. 8) if electrical coupling is capacitive or to a coil (not shown) if electrical coupling is inductive. Continuous or pulsed electrical discharges between said electrodes or within said coil complement the stimulated light emission treatment. Alternatively, a microwave guide could be used instead of said pair of electrodes or instead of said coil. If using plasma during stimulated light emission treatment, a certain restriction applies namely pressure inside chamber 10 should not exceed the value corresponding to the upper limit of plasma glow pressure range. Said range is typically between 10 millitorr and about one torr.

Processing of discrete objects 12 in the case when stimulated light emission treatment is effected after direct plasma treatment comprises the following steps: 1) load objects 12 into chamber 12; 2) turn vacuum pump 50 on, open valve 46 and evacuate chamber 10; 3) open valve 36 and turn power supply 76 on, then electrical discharges between electrodes 58 and 60 generate plasma inside chamber 10, said plasma treats objects 12; 4) turn power supply 76 off and turn power supply 84 on, then electrical discharges between electrodes 14 and 16 generate stimulate photons that further treat objects 12; 5) close valve 36 and turn power supply 84 off, then vacuum pump 50 evacuates chamber 10 into the atmosphere via valve 46, filter 48, scrubber 52 and vent 54; 6) open valve 38 and purge chamber 10 by an inert gas from container 32; 7) open vent valve 44 to break vacuum in chamber 10; 8) unload treated objects 12.

An example of said treatment is deposition of zinc, a p-type dopant onto a surface of silicon wafer. The deposition comprises the following steps.

Wafers 12 are loaded into chamber 10 onto perforated plate 22 wherein they are installed upright in cassettes (not shown). After loading is completed, door 26 is closed and sealed by Viton O-ring 28. Then vacuum pump 50 is turned on, valve 46 is open and evacuation of chamber 10 begins.

After evacuation is completed, valve 36 is open letting gaseous mixture from container 30 into chamber 10. Said gaseous mixture comprises carbon dioxide 9% by volume, nitrogen 13% by volume, dimethylzinc 0.05% by volume, hydrogen the rest. Opening of said valve governs the pressure inside chamber 10 maintaining it around 400 millitorr. Contents of chamber 10 are exhausted by vacuum pump 50 through valve 46, filter 48, scrubber 52 and vent 54.

Inside chamber 10 said mixture is subjected to electrical discharges between pair of electrodes 58 and 60 by turning on power supply 76. The discharges between electrodes 58 and 60 proceed with radio frequency of 13.56 megahertz, current of 1 ampere and voltage of 500 volt. Under said conditions hydrogen radicals are created out of hydrogen molecules present in said gaseous mixture. Said radicals abstract zinc from dimethyl zinc leaving methane as a byproduct while zinc deposits on the silicon wafer surfaces.

Deposition of zinc displaces silicon atoms in the wafer's lattice. As a result, silicon may partially lose its crystalline character. To recover crystallinity a wafer needs to be rapidly heated up. In order to achieve said rapid heating, power supply 84 is turned on after power supply 76 is turned off. Power supply 84 generates twenty kilovolt electrical discharges between electrodes 14 and 16. Said electrical discharges produce stimulated light emission in the infrared range with the wavelength of 10.6 micrometers. Infrared photons upon impingement on silicon surface rapidly heat it up. Additional conventional radiation sources (not shown) could also be employed for rapid wafer heating.

After treatment has been completed, chamber 10 is purged by opening valve 38 and letting nitrogen from tank 32 in. After purging, vacuum pump 50 is turned off, valve 46 is closed, and vacuum is broken by opening vent valve 44. Then door 26 is open and treated wafers 12 are unloaded.

The combination of stimulated light emission treatment with remote plasma treatment is realized by transporting plasma activated gas from plasma chamber 62 to chamber 10 wherein discrete objects 12 are subjected to stimulated light emission and reactions with chemical radicals. Some of said radicals are transported from chamber 62 while others are created in chamber 10.

Plasma chamber 62 could employ electrical discharges in radio or audio frequency range or comprise a microwave waveguide. Said chamber could be purchased from a plasma treatment equipment manufacturer such as Applied Materials, Veeco Instruments, LAM Research, March Instruments, Gasonics, etc.

Plasma chamber 62 could be connected to its own gas container 68 or alternatively to gas container 30. In the latter case both the plasma generation chamber 62 and the main treatment chamber 10 use the same gas or gaseous mixture.

Pipe 66 connecting the plasma chamber 62 and the main treatment chamber 10 should be short enough so as to allow most of the chemical radicals generated in chamber 62 to survive en route to chamber 10 while electrons and ions recombine.

Processing of objects 12 combining remote plasma treatment and stimulated light emission treatment comprises the following steps: 1) loading objects 12 into chamber 10; 2) turning vacuum pump 50 on, opening valves 122 and 46 and evacuating both plasma treatment chamber 62 and main treatment chamber 10; 3) supplying gas or gaseous mixture from container 68 or from container 30 to plasma chamber 62; 4) turning power supply 154 on, electrical discharges between electrodes 148 and 160 generate plasma glow in gas or gaseous mixture flowing through chamber 62; 5) closing valve 122 and opening valve 64 to allow flow of the plasmatized gas or gaseous mixture from chamber 62 to chamber 10 via pipe 66; 6) closing valves 46 and 64, turning pump 82 on, opening valves 36, 78 and 122 forcing gas flow through chamber 10 and generating stimulated light emission in said chamber by turning power supply 84 n so objects 12 are treated by chemical radicals created in both plasma chamber 62 and chamber 10 while plasma chamber 62 is simultaneously evacuated by vacuum pump 50 through open valve 122, filter 48, scrubber 52 and vent 54; 7) opening valve 46 starting evacuation of chamber 10 into the atmosphere by vacuum pump 50 through filter 48, scrubber 52, and vent 54; 8) purging chamber 10; 9) breaking vacuum in chamber 10; 10) unloading treated objects 12.

An example of stimulated light emission treatment combined with remote plasma is a process of improving bondability of polytetrafluoroethylene film. Polytetrafluoroethylene is an exceptionally inert and hydrophobic material. However, because of its inertness it is very difficult to bond tetrafluoroethylene with another material while in many applications it is advantageous to have the ability of bonding polytetrafluoroethylene to a device to improve inertness of said device.

For example, ink jet printer head could be covered with a polytetrafluoroethylene film to prevent ink from spreading over said head after ejection of an ink drop. At printing a drop is formed by heating ink and then the drop is ejected. If surface of ink jet head is not sufficiently hydrophobic, said drop after ejection may partially spread over said surface and subsequently the drop may travel to media where printing takes place, e.g. to paper, in unpredictable fashion leading to inferior quality of printing. If said surface is highly hydrophobic spreading of ink is checked and ink drops travel in predictable manner.

One of the methods to attain sufficient hydrophobicity of ink jet printer head is to attach a piece of polytetrafluoroethylene film to its surface. It could be done if one side of said piece is rendered bondable to the ink jet printer head surface while the other side of the piece remains hydrophobic.

The method of rendering one side of polytetrafluoroethylene film bondable to ink jet printer head surface comprises the following steps. The first step is loading pieces 12 of polytetrafluoroethylene film into chamber 10 onto plate 22 between perforations 24 in such a way that the film's side to be bonded faces upward. After loading, door 26 is closed and sealed by Viton O-ring 28. Then vacuum pump 50 is turned on, valves 46 and 122 are open, and evacuation of chambers 10 and 62 begins.

After evacuation is completed, valve 70 is open letting hydrogen from container 68 into plasma chamber 62. Mass flow controller 72 maintains pressure of 300 millitorr inside chamber 62 with flow rate of 100 sccm. An sccm means standard cubic centimeter per minute. The word “standard” in the above definition implies that sccm is the number of cubic centimeters of compressed gas under a certain pressure drop and with a certain temperature that would pass through chamber 62 in a minute if said pressure drop and said temperature were converted to the standard pressure drop of 1 atmosphere or 101,325 pascal and to the standard temperature of 0° C. or 273.16 kelvin.

When hydrogen from container 68 flows through chamber 62 it is subjected to electrical discharges between electrodes 148 and 160 with frequency of 13.56 megahertz. During discharges a portion of hydrogen molecules decomposes into hydrogen radicals.

After opening valve 64, hydrogen molecules and radicals together with ions and electrons flow through pipe 66 into chamber 10. During travel through said pipe most of electrons and ions recombine but said hydrogen radicals having longer lifetimes reach chamber 10. Herein said hydrogen radicals abstract fluorine from surfaces of polytetrafluoroethylene coupons. As Dr. Inagaki teaches in his monograph “Plasma Surface Modification and Plasma Polymerization”, Technomic Publishing Company, 1996, the remote hydrogen plasma treatment is more effective in improvement of the polytetrafluoroethylene bondability than the direct plasma treatment.

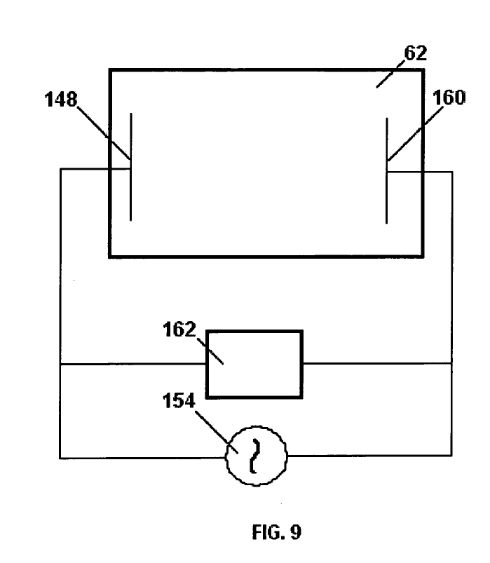

After a few minutes of remote hydrogen plasma treatment, plasma matching network 162 and power supply 154 (see FIG. 9) are turned off, valve 64 is closed while valve 122 is continues to be open and vacuum pump 50 exhausts hydrogen from plasma chamber 62. After completion of evacuation, valve 38 is open and chamber 62 is purged by nitrogen from container 32. After purging is completed, valve 122 is closed.

Gaseous mixture placed in container 30 comprises argon 9% by volume, tetrafluoromethane 20% by volume, hydrogen the rest. After valve 64 has been closed, pump 82 is turned on, valves 36 and 78 are open letting gaseous mixture from container 30 to circulate through chamber 10 via filtrer 42. The composition of the gaseous mixture in container 30 practically does not change because the quantities of hydrogen coming from chamber 62 are minute if compared with the quantity of hydrogen kept in said container.

When said gaseous mixture flows through chamber 10 it is subjected to twenty kilovolt electrical discharges between electrodes 14 and 16. Said discharges are delivered in pulses. Each pulse is 20 nanosecond to 40 nanosecond long. The frequency of pulses is from 10 hertz to 2 kilohertz.

Under high voltage discharges a portion of tetrafluoromethane decomposes into carbon and fluorine radicals. Said fluorine radicals combine with argon forming argon fluoride dimers. Said argon fluoride dimers produce photons of stimulated ultraviolet light emission with wavelength of 193 nanometers.

Said stimulated photons move back and forth bouncing off mirrors 18 and 20. Upon collisions with said stimulated photons hydrogen molecules dissociate into hydrogen radicals. Said hydrogen radicals together with remaining remote plasma hydrogen radicals replace fluorine in the exposed polytetrafluorethylene surfaces enhancing bondability of said surfaces.

After a certain number of pulses for example after 30,000 pulses power supply 84 and pulse generator 80 are turned off, valve 36 is closed. Because valve 78 is still open pump 82 pulls most of said gaseous mixture back to container 30.

Then valve 78 is closed, pump 82 is turned off, vacuum pump 50 is turned on, valve 46 is open, and evacuation of chamber 10 continues. After evacuation of chamber 10 is completed, valve 38 is open and said chamber is purged by nitrogen from tank 32. Then vacuum in chamber 10 is broken by opening vent valve 44, door 26 is open, and treated pieces 12 of polytetrafluorethylene film are unloaded.

Those knowledgeable in the art of materials processing understand that there could be numerous combinations of stimulated light emission treatments realized by using various gaseous mixtures sequentially and/or combining stimulated light emission treatment with plasma treatment. Some treatment steps could be followed by monomer deposition. Also, gaseous mixtures that generate stimulated light emission at various wavelengths could be employed each wavelength producing a separate desired effect.

Most of the embodiments of the apparatus for stimulated light emission treatment of discrete objects described above have an optical resonator typically formed by opposite concave mirrors 18 and 20 installed inside the main treatment chamber 10. Such an arrangement requires a trade-off for the pressure of gas or gaseous mixture flowing inside chamber 10 during materials processing. Said gas pressure should be optimal for stimulated light emission generation. On the other hand, said pressure should be also optimal for surface modification in a sense that mean free molecular path of excited species should be large enough so said species could reach treated surfaces without leaving the treatment chamber. Sometimes such a trade-off is difficult to achieve. In those cases it would be beneficial to enclose an optical resonator and place the enclosure inside the treatment chamber.

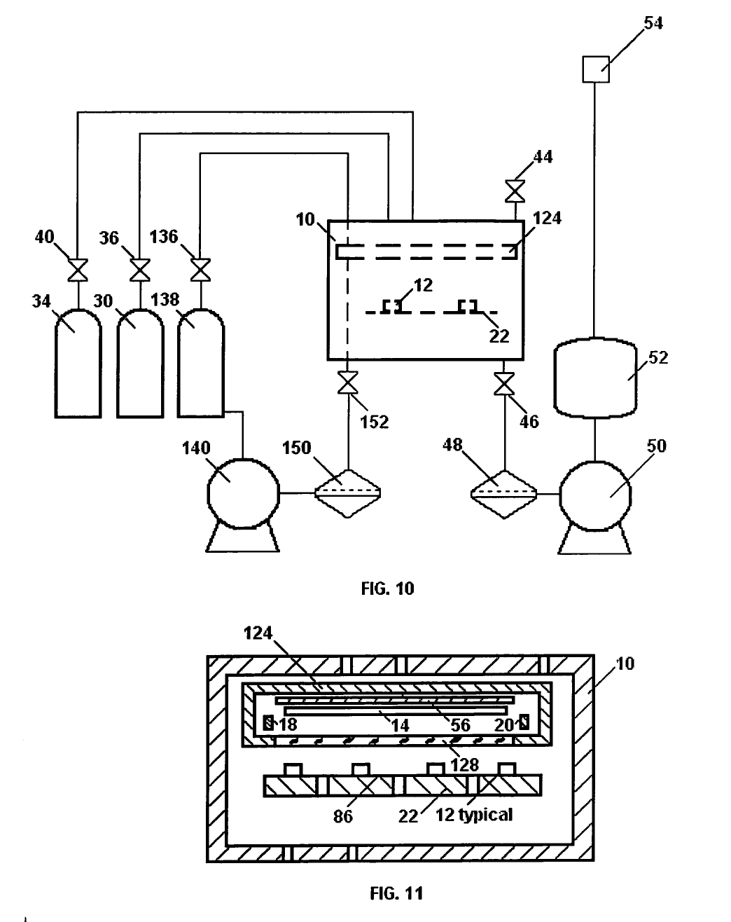

A schematic diagram of the apparatus for stimulated light emission treatment of discrete objects 12 using enclosure 124 is shown in FIG. 10. A frontal cross-sectional view of enclosure 124 is presented in FIG. 11.

A pump or fan 140 circulates gas or gaseous mixture capable of producing a population inversion from container 138 through enclosure 124. If there is no need for gas renewal said enclosure might be prefilled with said gas or gaseous mixture. If this is the case means for gas circulation through enclosure 124 (container 138, pump 140, filter 150, valve 152, and correspondent piping) are no longer needed.

Enclosure 124 comprises electrodes 14 and 16 and concave mirrors 18 and 20. The material of window 128 which is situated opposite discrete objects 12 should be capable of transmitting stimulated photons that escape an unstable optical resonator formed by concave mirrors 18 and 20. If the wavelength of stimulated photons is in the ultraviolet range said material could be the UV grade calcium fluoride, CaF₂. Also, a fully reflective mirror 56 is installed above mirrors 18 and 20 opposite window 128 to redirect stimulated photons through said window back to objects 12. Instead of mirror 56, inner walls of enclosure 124 could be shaped and treated in such a manner as to make them reflective of photons generated by stimulated light emission.

Enclosure 124 comprising electrodes 14 and 16, mirrors 18, 20 and 56 and transparent window 128 differs from conventional laser in three important aspects.

First, both concave mirrors 18 and 20 are fully reflective while in a laser one mirror is partially reflective to let coherent light beam out.

Second, mirrors 18 and 20 form an unstable resonator that allows stimulated photons eventually escape through transparent window 128 and subsequently promote useful chemical reactions in the flowing gas and on treated surfaces of objects 12 while a laser typically employs a stable optical resonator.

Third, a fully reflective mirror 56 is installed opposite a transparent window 128 or inner walls of enclosure 124 are shaped and/or treated in such a manner as to redirect stimulated photons through said window towards objects 12.

Target plate 130 covered with volatile material (not shown) could also be installed under transparent window 128. Typically it may be installed when gas pressure inside chamber 10 corresponds to high vacuum. In high vacuum molecules volatilized due to collisions of target coating with stimulated photons travel fast and may deposit on surfaces of objects 12. If the gas pressure inside chamber 10 is too high said molecules may not reach said surfaces during the treatment time.

The materials processing by stimulated light emitted from enclosure 124 is accomplished by the following steps: 1) objects 12 are loaded into chamber 10; 2) chamber 10 is evacuated by vacuum pump 50; 3) while vacuum pump 50 continues to be on valve 36 is open, gas or gaseous mixture from container 30 starts flowing through chamber 10, simultaneously stimulated light emission is generated inside enclosure 124, stimulated photons passing through transparent window 128 break bonds in molecules of the flowing gas and on the treated surfaces of objects 12 creating species like chemical radicals, excited atoms and molecules that modify said surfaces; 4) chamber 10 is evacuated after said treatment has been completed; 5) purging chamber 10; 6) breaking vacuum in chamber 10; 7) unloading treated objects 12.

An example of stimulated light emission treatment using enclosure 124 is etching of silicon oxide. As a part of semiconductor chip manufacturing process, impurities like phosphorus are introduced into silicon lattice to increase conductivity. Silicon oxide layer is grown over silicon surface to provide a barrier to said impurities during their deposition. Then organic photoresist is applied over said silicon oxide layer. Using photolithography processing, portions of the photoresist are removed to uncover the areas for impurities deposition. In said uncovered areas silicon oxide should be etched. The etching processing steps are as follows.

Container 30 is filled with gaseous mixture of tetrafluoromethane 96% by volume and oxygen 4% by volume. Gaseous mixture in container 138 comprises argon 9% by volume, fluorine 0.2% by volume, neon 25% by volume, helium the rest. Because fluorine has tendency to be depleted with time due to its high reactivity, said mixture's composition is maintained by circulation accomplished by means of centrifugal fan 140. The gaseous mixture's pressure inside enclosure 124 is maintained at about four atmospheres. Material of enclosure 124, container 138, valves 136 and 152, filter 150, and all pipes and fittings exposed to said gaseous mixture is stainless steel with nickel coating capable of withstanding high reactivity of fluorine.

Wafers 12 covered with a mask of developed photoresist are loaded into chamber 10 and the chamber is evacuated. After valve 36 is open, gaseous mixture from container 30 starts flowing through chamber 10. Valve 36 is adjusted in such a way as to maintain pressure in chamber 10 at about 100 millitorr.

Simultaneously with opening of valve 36 power supply 84 and pulse generator 0 are turned on generating stimulated light emission with the wavelength of 193 anometers between electrodes 14 and 16 inside enclosure 124. Stimulated photons with the wavelength of 193 nanometers that escape enclosure 124 through window 128 possess energy of 620 kilojoule/mole. Said energy is higher than the energy of carbon—fluorine bond (441 kilojoule/mole). Therefore, said stimulated photons break carbon—fluorine bonds in tetrafluoromethane molecules releasing fluorine atoms. Said fluorine atoms react with silicon oxide at exposed areas of wafers 12 creating volatile products (mainly silicon tetrafluoride, SiF₄) that are carried away by vacuum pump 50.

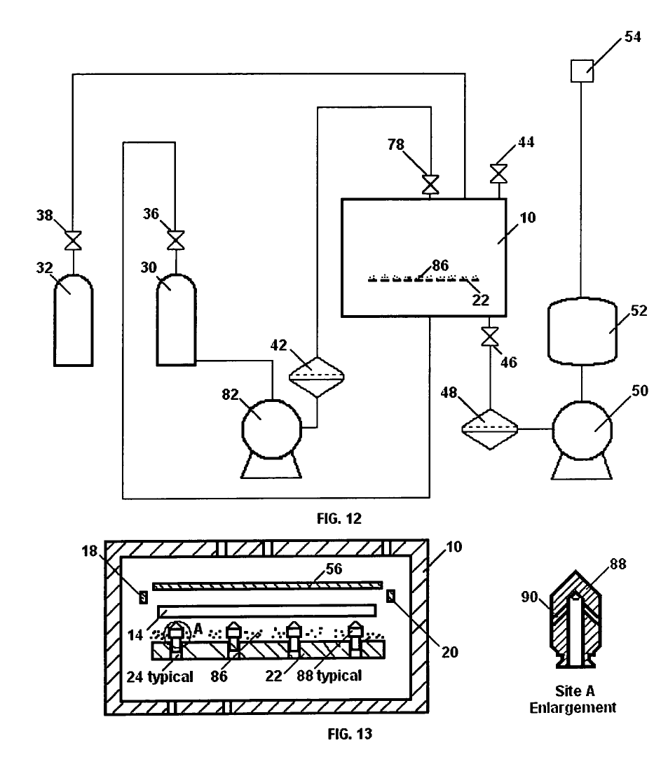

The schematic diagram of the apparatus for stimulated light emission treatment of powder-like materials 86 such as pellets, granules, beads is shown in FIG. 12. Frontal cross-sectional view of chamber 10 employed for treatment of powder-like materials is shown in FIG. 13.

Hereinbelow for the sake of brevity let us call pellets, granules, beads and other particulate matter 86 treated by the method of the present invention as particles. The gas pressure in chamber 10 and other conditions inside chamber 10, e.g. the design of support means 22, should provide for fluidization of particles 86. Fluidization means said particles levitate in gas flow. Said levitation occurs when gas speed reaches the velocity at which the force exerted by the flowing gas on a particle is equal to the force of gravity. At a given pressure drop such velocity is determined by particle sizes and sizes of perforations 24 in support means 22 or if said means comprise a porous plate such velocity at a given pressure drop is determined by particle sizes and pore sizes. In order to prevent particle weeping back through perforations 24 inserts 88 could be installed in support means 22 said inserts having inclined channels 90. The processing of particles according to the method of the present invention is as follows.

First, a layer of particles 86 is loaded onto support means 22. Then door 26 is closed, vacuum pump 50 is turned on, valve 46 is open, and chamber 10 is evacuated. After evacuation is completed, valve 46 is closed, vacuum pump 50 is turned off, valves 36 and 78 are open, pump 82 is turned on, and gas or gaseous mixture from container 30 starts circulating through chamber 10.

After the onset of fluidization the power supply 84 and pulse generator 80 (FIG. 4) are turned on and electrical discharges between electrodes 14 and 16 start generating stimulated light emission in the flowing gas.

Stimulated photons hit fluidized particles creating radicals on their surfaces. Also, stimulated photons create radicals in the flowing gas. Said gas radicals drift to the surfaces of the fluidized particles and create useful chemical functional groups by reacting with surface radicals.

The characteristics of pump 82 are selected in such a way as to make sure the gas velocity after exiting channels 90 fluidizes particles 86. Another requirement is that the pressure of the gas exiting channels 90 should be equal to the pressure necessary for generation of stimulated light emission during electrical discharges. Yet another requirement is that the diffusion rate of chemical radicals created by collisions of stimulated photons with gas molecules be sufficient for said radicals to reach surfaces of treated particles.

After the treatment is completed, power supply 84 and pulse generator 80 are turned off, particles 86 settle down on support means 22, valve 36 is closed while valve 78 is kept open letting gas to be evacuated to container 30 by pump 82 through filter 42.

After said evacuation is completed, valve 78 is closed, pump 82 is turned off, vacuum pump 50 is turned on, valve 46 is open. After chamber 10 is fully evacuated, it is purged by an inert gas from container 32. Then vacuum is broken by opening vent valve 44. After that door 26 is open and treated particles 86 are unloaded.

An example of particle treatment by stimulated light emission is surface modification of ion exchange beads for water treatment. Water typically contains positively charged cations: calcium, magnesium, sodium, etc. Said cations deleteriously affect surfaces of industrial systems such as heating and cooling equipment, steam generators, etc. Negatively charged ion exchange beads pull said cations out of water and thus improve the performance of said industrial systems.

A layer of 90% polystyrene/8% divinylbenzene/2% benzophenone ion exchange beads 86 with average diameter of twenty micrometers is placed on support plate 22. After evacuation of chamber 10 pump 82 starts moving gaseous mixture from container 30 through said chamber. Said gaseous mixture comprises argon 9% by volume, sulfur hexafluoride 16% by volume, sulfur dioxide 5% by volume, tetrafluorohexane 0.1% by volume, helium the rest. The pressure of gaseous mixture after valve 36 should be sufficient to ensure the gas velocity fluidizes beads 86.

When power supply 84 and pulse generator 80 are on, electrical discharges between electrodes 14 and 16 generated in ten nanosecond pulses with the pulse frequency of one hundred hertz break sulfur hexafluoride molecules creating fluorine radicals. Said radicals combine with argon during discharges forming argon fluoride dimers. After an electrical discharge between electrodes 14 and 16 is over said dimers being unstable deexcite to the ground state emitting stimulated photons with the wavelength of 193 nanometers, i.e. having energy of 620 kilojoule/mole. If a non-linear crystal is installed between mirrors 18 and 20 there is a mix of stimulated photons, some with the energy of 620 kilojoule/mole and others with the energy of 1,240 kilojoule/mole.

Upon impingement of said stimulated photons on the bead surface, polystyrene chains are broken and chemical radicals are formed at scission sites. Simultaneously said stimulated photons break gas molecules forming gas radicals. Said gas radicals drift to fluidized beads and react with radicals at polystyrene chain scission sites. As a result bead surface becomes highly fluorinated with sulfonic and carboxyl functional groups present. Such surface is ideal for cation exchange.

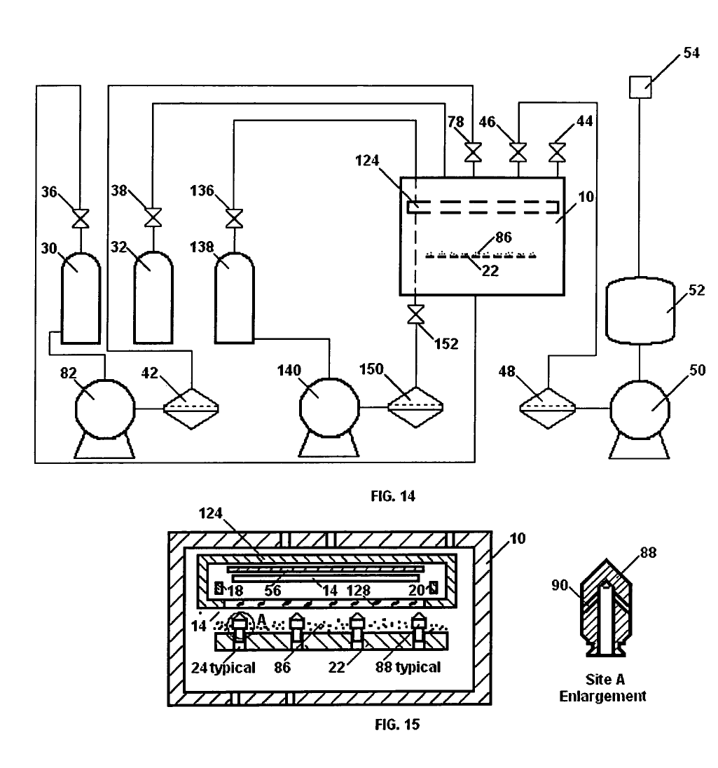

The apparatus for stimulated light emission treatment of particles may include enclosure 124 as shown in FIG. 14. A frontal cross-sectional view of the enclosure is presented in FIG. 15.

In the case of using enclosure 124 gas or gaseous mixture that fills container 30 does not need a population inversion capability though it might have it. Also, pressure constraints during processing are less stringent because a trade-off between pressure necessary to achieve a population inversion and pressure allowing chemical radicals generated by stimulated light emission to move swiftly to particle surfaces is no more required.

The powder-like materials processing by stimulated light emitted from enclosure 124 comprises the following steps: 1) particles 86 are loaded onto holding means 22 in chamber 10; 2) vacuum pump 50 is turned on, valve 46 is open, and chamber 10 is evacuated; 3) after evacuation is completed, valve 46 is closed, vacuum pump 50 is turned off, pump 82 is turned on, valves 36 and 78 are open so gaseous mixture from tank 30 circulates through chamber 10 fluidizing particles 86, simultaneously power supply 84 (FIG. 4) is turned on, electrical discharges between electrodes 14 and 16 generate stimulated emission in gaseous mixture supplied from tank 138, said gaseous mixture circulates through enclosure 124 by means of pump 140, stimulated photons passing through transparent window 128 break bonds in molecules of gaseous mixture from tank 30 creating chemical radicals that drift to the surfaces of fluidized particles 86 and react there forming useful chemical groups on said surfaces; 4) after treatment is completed, power supply 84 is turned off, valve 36 is closed, and pump 82 pulls remaining gaseous mixture back to tank 30; 5) after that pump 82 is turned off, valve 78 is closed, vacuum pump 50 is turned on, valve 46 is open and chamber 10 is fully evacuated; 6) after evacuation, vacuum pump 50 continues to be on, valve 38 is open and chamber 10 is purged by an inert gas from container 32; 7) after purging, vacuum is broken by opening vent valve 44, then door 26 is open and treated particles 86 are unloaded.

Using enclosure 124 for coating of the 90% polystyrene/8% divinylbenzene/2% benzophenone ion exchange beads 86 container 138 is filled with the gaseous mixture comprising argon 9% by volume, fluorine 0.2% by volume, neon 25% by volume, helium the rest. Container 30 is filled with the gaseous mixture comprising sulfur dioxide 5% by volume, tetrafluorohexane 0.1% by volume, helium the rest.

After loading beads 86 onto support plate 22 and evacuating chamber 10, valves 36 and 78 are open, pump 82 is turned on and starts circulation of gaseous mixture from container 30 through chamber 10. Simultaneously, power supply 84 and pulse generator 80 (FIG. 4) are turned on. Stimulated photons with the wavelength of 193 nanometers generated inside enclosure 124 get into chamber 10 through window 128. Said stimulated photons break bonds in gas molecules and in bead polystyrene chains promoting bead surface fluorination and grafting of sulfonic and carboxyl functional groups.

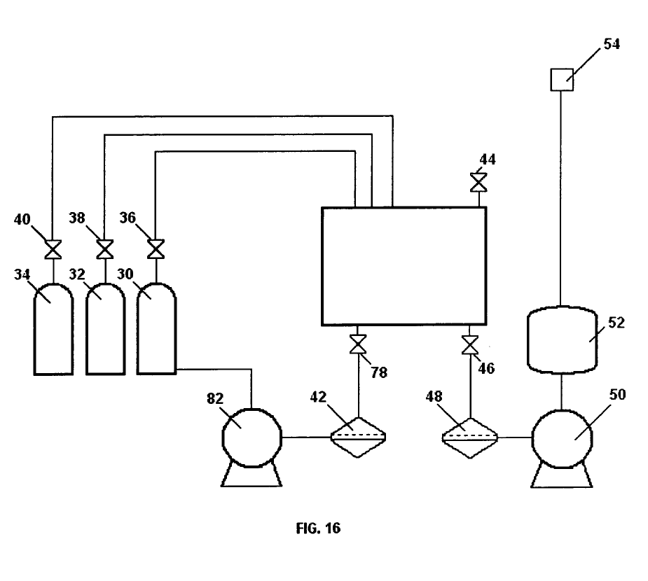

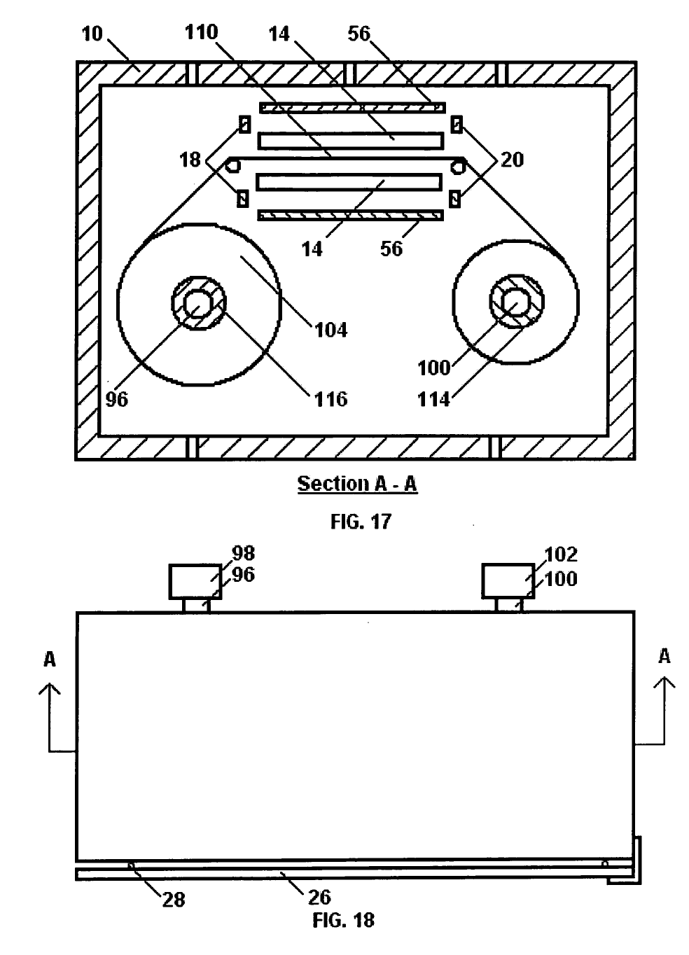

In many materials processing applications it is necessary to treat continuous media such as films, webs, cables or wires. Examples are rolls of polyethylene films for packaging, polypropylene membranes for catching bacteria and viruses, optical fibers. Schematic diagram of apparatus for such application is shown in FIG. 16. Views of the treatment chamber 10 utilized for processing of continuous media are presented in FIG. 17 and FIG. 18.

Processing of continuous media comprises the following steps: 1) roll 104 preinstalled on core 116 is loaded into the treatment chamber 10 and said core is secured on the take-off shaft 96, then media 110 is pulled onto core 114 and is secured there the core 114 being preinstalled on take-on shaft 100; 2) vacuum pump 50 is turned on, valve 46 is open, and chamber 10 is evacuated; 3) after evacuation is completed, valve 46 is closed, vacuum pump 50 is turned off, pump 82 is turned on, valves 36 and 78 are open letting compressed gas from container 30 to circulate through chamber 10, simultaneously motor 102 is turned on unwinding roll 104, and power supply 84 and pulse generator 80 (FIG. 4) are turned on generating electrical discharges between electrodes 14 and 16, said discharges produce stimulated light emission, stimulated photons break bonds in molecules of circulating gaseous mixture and in molecules on the surfaces of media 110 promoting chemical reactions that advantageously modify said surfaces; 4) after media 110 has been rewound onto core 114 but its end is still secured in core 116, valve 36 is closed, power supply 84 and pulse generator 80 are turned off, pump 82 continues to be on and evacuates contents of chamber 10 back to container 30; 5) valve 78 is closed, pump 82 is turned off, vacuum pump 50 is turned on, valve 46 is open, and chamber 10 is fully evacuated; 6) after evacuation is completed, valve 38 is open and chamber 10 is purged by an inert gas from container 32; 7) vacuum is broken by opening vent valve 44, door 26 is open and treated roll 104 is unloaded.

The above described processing could be complemented by monomer deposition. A part of radicals created on surfaces of media 110 during stimulated light emission treatment do not combine with gaseous mixture excited species. In order to use said surface radicals the following steps are added to the above described processing. Step 3 is modified so that pump 82 evacuates chamber 10 until pressure inside said chamber becomes less than vapor pressure of monomer kept in container 34. After said pressure is reached, pump 82 is turned off, valve 78 is closed, valve 40 is open, motor 102 is turned off, motor 98 is turned on rotating shaft 96 in the direction opposite to the direction of the rotation of shaft 100, and media 110 is being wound back onto core 116 while monomer vapor from container 34 reacts with remaining excited species at scission sites on surfaces of said rewinding media. After media 110 is completely rewound onto core 116, motor 98 is turned off, valve 40 is closed, vacuum pump 50 is turned on, valve 46 is open and chamber 10 is evacuated. After evacuation is completed, valve 38 is open and chamber 10 is purged by an inert gas from container 32. After purging, vacuum is broken by opening vent valve 44, door 26 is open, and treated roll 104 is unloaded.

An example is surface modification of polyethylene film from which packaging bags are made. Said film is required to possess a barrier capability against ambient moisture. It means that polyethylene surface should be sufficiently hydrophobic to be able to repel penetration of condensed water through pores into the bag.

Gaseous mixture in container 30 comprises argon 9% by volume, sulfur hexafluoride 29% by volume, helium the rest. When power supply 84 and pulse generator 80 (FIG. 4) are on, electrical discharges between electrodes 14 and 16 break sulfur hexafluoride molecules creating fluorine radicals. Said radicals combine with argon during discharges forming argon fluoride dimers. After an electrical discharge between electrodes 14 and 16 is over, said dimers being unstable deexcite to the ground state emitting stimulated photons with the wavelength of 193 nanometers, i.e. having energy of 620 kilojoule/mole. If a non-linear crystal is installed between mirrors 18 and 20 there is a mix of stimulated photons, some with the energy of 620 kilojoule/mole and others with the energy of 1,240 kilojoule/mole. Motor 102 is turned on and starts pulling polyethylene film 110 from core 116 to core 114. While moving, polyethylene film 110 is subjected to impingement of photons with said energies causing scissions of carbon-carbon and carbon-hydrogen bonds.

Between pulses fluorine radicals displace hydrogen at scission sites. Said fluorine incorporation significantly reduces surface energy rendering treated surfaces hydrophobic. Said process continues until all treated polyethylene film 110 is wound onto core 114 still being attached to the core 116.

Then power supply 84 and pulse generator 80 are turned off and chamber 10 is evacuated to the gas pressure less than 1.5 torr at 20° C., vapor pressure of cyclooctamethylsiloxane, a hydrophobic monomer placed in container 34. When said pressure value is reached, valve 40 is open letting vapor of cyclooctamethylsiloxane from container 34 into chamber 10. Motor 102 is turned off, motor 98 is turned on starting reverse movement of polyethylene film 110 back onto core 116. During said reverse movement surfaces of treated material are exposed to hydrophobic cyclooctamethylsiloxane vapor. Cyclooctamethylsiloxane molecules polymerize via radical polymerization at remaining scission sites on the treated surfaces enhancing hydrophobicity even further. After all polyethylene film is rewound onto core 116, chamber 10 is evacuated and purged with nitrogen from container 32. Then vacuum is broken by opening vent valve 44 and treated roll 104 is unloaded.

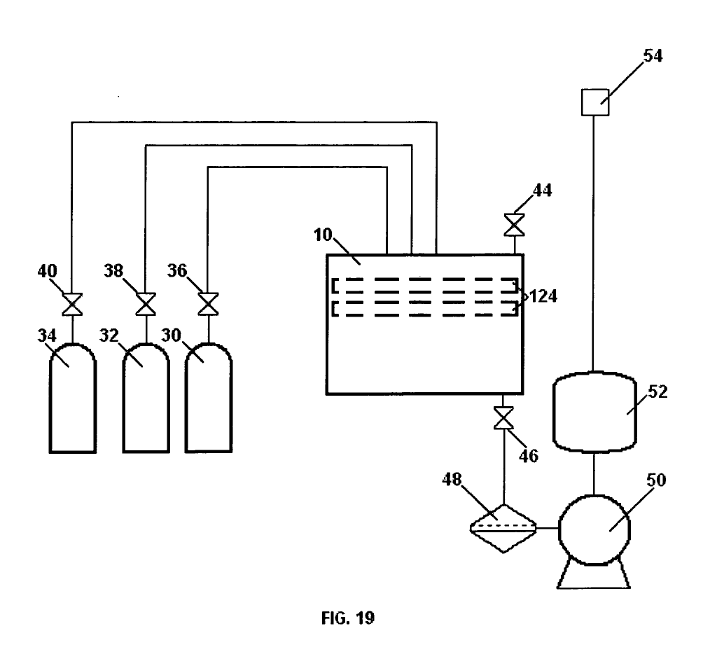

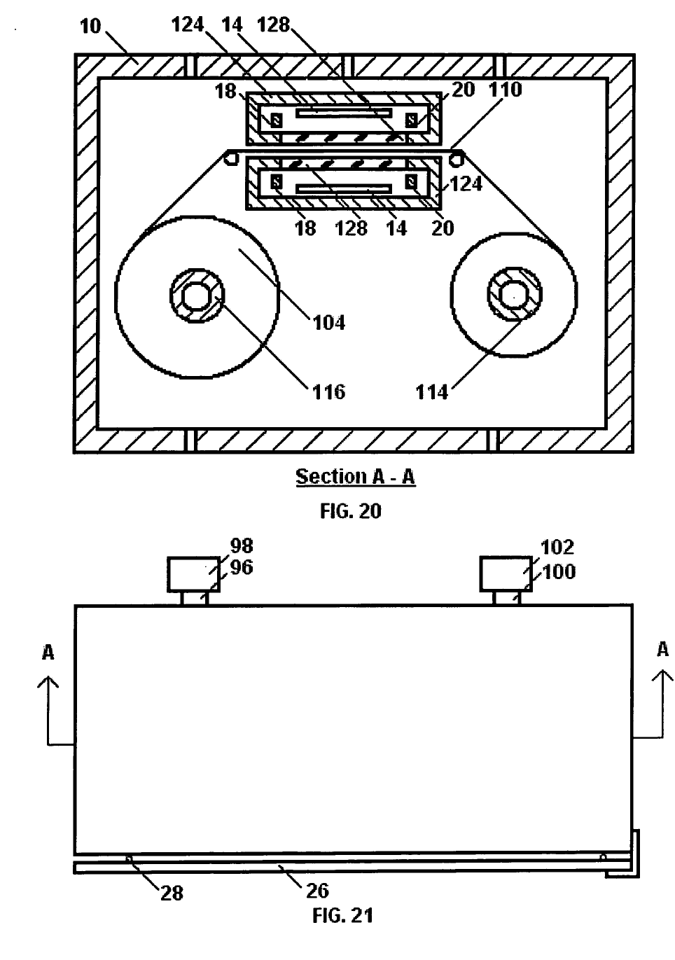

The continuous media processing could be performed with electrodes 14 and 16 and mirrors 18, 20 and 56 placed inside enclosure 124. Schematic diagram of the apparatus for such processing is shown in FIG. 19. Views of treatment chamber 10 utilized for processing of continuous media using enclosures 124 are presented in FIG. 20 and FIG. 21.

The steps for processing of continuous media using enclosures 124 are similar to those described above for processing of continuous media without said enclosures. However, by contrast, gas or gaseous mixture flowing through chamber 10 the lafter having enclosures 124 does not need to have a population inversion capability and as a corollary to this gas pressure requirements inside chamber 10 are less stringent than in the case when there is no enclosures.

Stimulated light emission processing of continuous media using enclosures 124 comprises the following steps: 1) roll 104 preinstalled on core 116 is loaded into the treatment chamber 10 with said core being secured on take-off shaft 96, then media 110 is pulled and secured on core 114 said core being preinstalled on take-on shaft 100; 2) vacuum pump 50 is turned on, valve 46 is open, and chamber 10 is evacuated; 3) while vacuum pump 50 continues to be on valve 36 is open, gas or gaseous mixture from container 30 starts flowing through chamber 10, simultaneously motor 102 is turned on starting to unwind roll 104 while power supply 84 and pulse generator 80 (FIG. 4) are turned on generating stimulated light emission treatment inside enclosure 124, stimulated photons pass through transparent window 128 and break bonds in molecules of the flowing gas and on the surfaces of media 110; 4) after media 110 has been rewound onto core 114 but its end is still secured in core 116, power supply 84 and pulse generator 80 are turned off, valve 36 is closed, vacuum pump 50 continues to be on and evacuates contents of chamber 10 via filter 48, scrubber 52 and vent 54 into the atmosphere; 5) after evacuation is completed, valve 38 is open and chamber 10 is purged by an inert gas from container 32; 6) vacuum is broken by opening vent valve 44, door 26 is open and treated roll 104 is unloaded.

The above described processing could be complemented by monomer deposition through modifying step 4 so that vacuum pump 50 evacuates chamber 10 until pressure inside said chamber becomes less than the vapor pressure of monomer kept in container 34 and adding the following steps after the modified step 4: 5) after said pressure is reached, valve 46 is closed, vacuum pump 50 is turned off, valve 40 is open, motor 102 is turned off, motor 98 is turned on, and media 110 is being wound back onto core 116 while monomer vapor from container 34 deposits onto surfaces of media 110; 6) after said media is completely rewound onto core 116, motor 98 is turned off, valve 40 is closed, vacuum pump 50 is turned on, valve 46 is open, and residual monomer vapor is evacuated; 7) after evacuation is completed, valve 38 is open and chamber 10 is purged by an inert gas from container 34; 8) after purging, vacuum is broken by opening vent valve 44, door 26 is open, and treated roll 104 is unloaded.

An example is hydrophilization of Nuclepore membrane. Nuclepore is a polycarbonate film having pores produced commercially with irradiation of said film by fission fragments of uranium isotope 235 and subsequent etching. It is used for precision filtration in electronics, chemical, biomedical and food industries because pore sizes are very uniform. Polycarbonate is hydrophobic, so it has to be hydrophilized in order to be effectively used for filtration.

A roll 104 of polycarbonate Nuclepore media is loaded into chamber 10 and the chamber is evacuated by vacuum pump 50. While said vacuum pump is still on, motor 102 is turned on starting to unwind the media and simultaneously valve 36 is open, and power supply 84 together with pulse generator 80 are turned on.

Enclosure 124 is filled with krypton chloride, KrCl gas premix that could be obtained from commercial sources, e.g. from Spectra Gases. Electrical discharges inside enclosure 124 generate stimulated photons with the wavelength of 222 nanometers which is equivalent to the energy of 539 kilojoule/mole.

Container 30 is filled with gaseous mixture of nitrogen dioxide in equilibrium with its dimer, dinitrogen tetraoxide. After vacuum pump 50 is turned on, valve 46 and 36 are open, and gaseous mixture from container 30 starts flowing through chamber 10. Single nitrogen—oxygen bonds in said gaseous mixture's molecules have bond energy value of 175 kilojoule/mole which is less than stimulated photons' energy of 539 kilojoule/mole. Said stimulated photons break said single bonds producing oxygen radicals and nitric oxide. Also, stimulated photons create scission sites on the surfaces of moving polycarbonate media by breaking carbon-carbon bonds said bonds having the bond energy value of 344 kilojoule/mole which is less than photons' energy of 539 kilojoule/mole. Said oxygen radicals drift to the surfaces of moving polycarbonate media and attach at scission sites creating useful functional groups such as hydroxyl, carboxyl, and carbonyl groups that render said surfaces hydrophilic.

After media 110 is completely rewound onto core 114 still being attached to the core 116, power supply 84 and pulse generator 80 are turned off and chamber 10 is evacuated by vacuum pump 50 to the gas pressure less than 160 millitorr which is the vapor pressure of hydroxypropyl acrylate, a hydrophilic monomer placed in container 34. When said pressure value is reached, valve 40 is open letting vapor of hydroxypropyl acrylate from container 34 into chamber 10. Motor 102 is turned off, motor 98 is turned on starting reverse movement of polycarbonate media 110 back onto core 116. During said reverse movement surfaces of treated material are exposed to hydroxypropyl acrylate vapor. Hydroxypropyl acrylate molecules polymerize via radical polymerization at remaining scission sites on the polycarbonate surfaces enhancing hydrophilicity even further. After all polycarbonate media is rewound onto core 116, chamber 10 is evacuated and purged with nitrogen from container 32. Then vacuum is broken by opening vent valve 44 and treated roll 104 is unloaded.

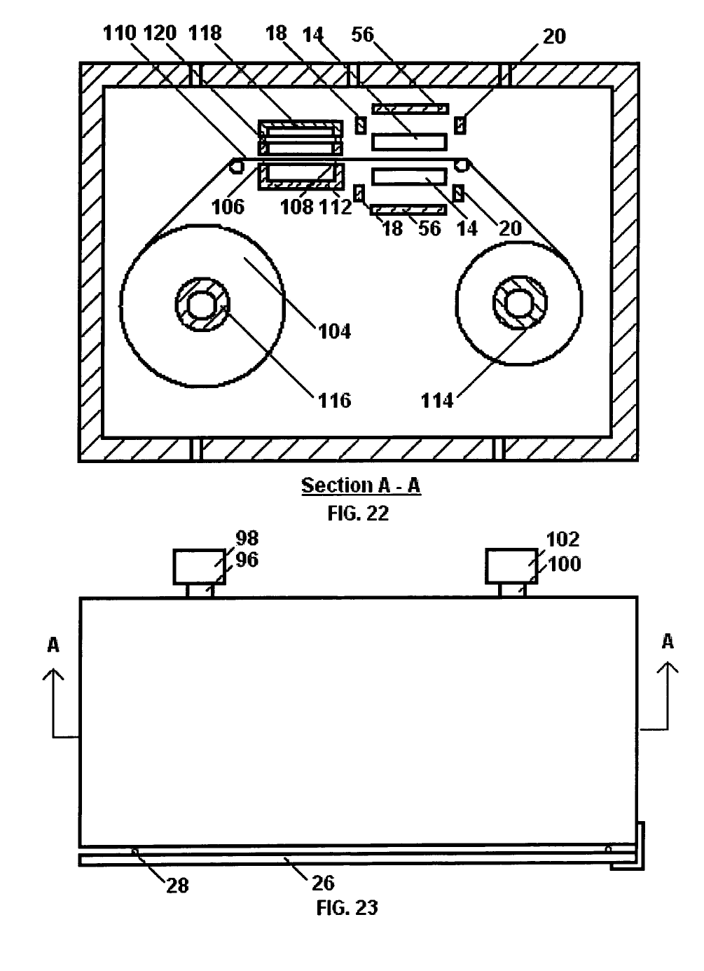

Continuous media often is precoated mostly by pulling said media through a bath filled with a suitable liquid. After leaving said bath, said precoated media is cured by radiation. Schematic diagram of apparatus for stimulated light emission treatment with media precoating is the same as without precoating (see FIG. 16). Frontal cross-sectional and bottom views of chamber 10 used in the apparatus of the present invention for treatment of continuos media by stimulated light emission with precoating are shown in FIG. 22 and FIG. 23 respectively.

Processing steps for stimulated light emission treatment with media precoating comprise the following steps: 1) roll 104 secured on core 116 is loaded into chamber 10 and said core is placed onto take-off shaft 96; 2) lid 118 of the bath 112 is open, bath 112 is filled with liquid used for said media precoating, media 110 is threaded through slits 106 and 108, secured on core 114, said core being preinstalled on take-on shaft 100, and lid 118 is closed and sealed by any suitable means, e.g. by Viton O-ring 120; 3) vacuum pump 50 is turned on, valve 46 is open, and chamber 10 is evacuated; 4) after evacuation of chamber 10 is completed, valve 46 is closed, vacuum pump 50 is turned off, pump 82 is turned on, valves 36 and 78 are open, and compressed gas or gaseous mixture from container 30 starts flowing through chamber 10, simultaneously motor 102 is turned on to unwind roll 104, and power supply 84 and pulse generator 80 (FIG. 4) are turned on starting stimulated light emission treatment of media 110; 5) said treatment continues until media 110 has been rewound onto core 114, then valve 36 is closed while pump 82 continues to be on evacuating contents of chamber 10 back into container 30, 6) valve 78 is closed, pump 82 is turned off, vacuum pump 50 is turned on, valve 46 is open, and chamber 10 is fully evacuated; 7) after evacuation, vacuum is broken by opening vent valve 44, door 26 is open, and treated roll 104 is unloaded.

An example is curing of acrylic coating of an optical fiber by stimulated light emission. Container 30 is filled with nitrogen. Roll 104 of optical fiber 110 is loaded into chamber 10 and threaded through slits 106 and 108. Bath 112 is

1. A materials processing apparatus for surface modification and/or cleaning, sterilization, disinfection, film deposition, and etching of discrete objects, powder-like substances and continuous media comprising
a treatment chamber with means for holding treated objects, means for generating stimulated light emission in a gas or a gaseous mixture capable of achieving a population inversion such as argon, neon, krypton, xenon, helium, nitrogen, carbon dioxide, carbon monoxide, hydrogen or gaseous mixtures thereof and means for reflecting stimulated light in the direction of treated objects;
at least one gas container;
means for moving gas or gaseous mixture through the treatment chamber.

2. A materials processing apparatus according to claim 1 wherein
means for generating stimulated light emission comprising at least one unstable optical resonator and means for reflecting stimulated light in the direction of treated objects are placed inside at least one enclosure said enclosure being filled with gas or gaseous mixture capable of achieving a population inversion or being connected to a container filled with gas or gaseous mixture capable of achieving a population inversion and to means of moving said gas or gaseous mixture through said enclosure;
a window transparent to the wavelength of stimulated light generated in the enclosure is placed in the enclosure's wall opposite means for holding treated objects inside the treatment chamber.

3. A materials processing apparatus according to claim 1 wherein means for generating stimulated light emission comprise at least one unstable optical resonator.

4. A materials processing apparatus according to claim 3 wherein a non-linear crystal is installed inside an unstable optical resonator.

5. A materials processing apparatus according to claim 3 wherein an unstable optical resonator comprises two opposite concave fully reflective mirrors.

6. A materials processing apparatus according to claim 1 wherein means covered with material that volatilizes under irradiation of stimulated photons are installed inside the treatment chamber.

7. A materials processing apparatus according to claim 1 wherein means for holding discrete treated objects or powder-like substances have perforations allowing free passage of gas or gaseous mixture around said objects.

8. A materials processing apparatus according to claim 7 wherein at a given pressure drop said perforations have sizes permitting fluidization of gas or gaseous mixture while said gas or gaseous mixture flows around powder-like substances placed onto the holding means.

9. A materials processing apparatus according to claim 7 wherein inserts preventing backward flow of powder-like substances are installed in the perforations and at a given pressure drop the sizes of orifices of said inserts permit fluidization of gas or gaseous mixture while said gas or gaseous mixture flows around powder-like substances placed onto the holding means.

10. A materials processing apparatus according to claim 1 wherein the treatment chamber is connected to a conventional plasma chamber the latter comprising a pair of electrodes connected to a power supply, or a coil connected to power supply, or a microwave guide.

11. A materials processing apparatus according to claim 1 wherein the treatment chamber is connected to at least one container filled with a reactive monomer such as allyl amine, vinyl acetate, allyl alcohol, styrene, acrylic acid, cyclooctamethylsiloxane, methacrylic acid, glycidyl methacrylate, hydroxypropyl acrylate, N-vinyl pyrrolidone, hydroxyethyl methacrylate, acrylamide.

12. A materials processing apparatus according to claim 1 wherein the object holding means have at least two cores installed on shafts and at least one of the shafts is connected to a motor said motor providing for rotation of continuous media exposed to stimulated light emission.

13. A materials processing apparatus according to claim 12 wherein the treatment chamber comprises a bath with precoating liquid said bath having means for ingress and egress of treated continuous media.

14. A materials processing method comprising the steps of:
loading discrete objects or powder-like substances onto object holding means in the treatment chamber of the apparatus according to claim 1;
evacuating the treatment chamber;
forcing gas or gaseous mixture to flow through the treatment chamber while the means for generating stimulated light emission are activated;
evacuating the treatment chamber after stimulated light emission activation has been shut off;
breaking vacuum in the treatment chamber;
unloading treated objects.

15. A materials processing method according to claim 14 including the steps of:
evacuating the treatment chamber and a conventional plasma chamber;
forcing gas or gaseous mixture through the plasma chamber and generating plasma glow there;
letting plasma activated gas or gaseous mixture to flow from the conventional plasma chamber into the treatment chamber while forcing same or another gas or gaseous mixture to flow through the treatment chamber wherein the means for generation of stimulated light emission are activated;
evacuating the conventional plasma chamber either simultaneously with the treatment in the treatment chamber or after the treatment in said chamber has been completed.

16. A materials processing method according to claim 14 including the step of letting active monomer vapor to flow into the treatment chamber after the stimulated light emission treatment step has been completed.

17. A materials processing method according to claim 14 including the step of forcing gas or gaseous mixture to flow through the treatment chamber in such a way as to ensure fluidization of the powder-like substances placed onto the object holding means while the means for generating stimulated light emission are activated.

18. A materials processing method comprising the steps of:
loading a roll of continuous media like films, webs, cables or wires in the treatment chamber of the apparatus according to claim 12;
evacuating the treatment chamber;
forcing gas or gaseous mixture to flow through the treatment chamber while the means for generating stimulated light emission are activated and simultaneously unwinding the media roll inside the treatment chamber while continuously exposing media to stimulated light emission generated either by gas or gaseous mixture flowing through the treatment chamber, or by gas or gaseous mixture filling the enclosure installed inside the treatment chamber, or by gas or gaseous mixture flowing through the enclosure installed inside the treatment chamber;
evacuating the treatment chamber after stimulated light emission activation has been shut off;
breaking vacuum in the treatment chamber;
unloading treated media.

19. A method according to claim 18 including the step of letting active monomer vapor to flow into the treatment chamber together with reverse unwinding of continuous media after the stimulated light emission treatment of said continuous media has been completed.

20. A method according to claim 18 including the step of precoating of continuous media prior to the start of stimulated light emission treatment.
